(19) United States
(12) Patent Application Publication
(10) Pub. No.: US 2017/0290685 A1
(43) Pub. Date: Oct. 12, 2017

(54) Advanced Fitment of Prosthetic Devices

(71) Applicant: Texas Research International, Inc., Austin, TX (US)

(72) Inventors: Jake Parker Montez, San Antonio, TX (US); David S. Forsyth, Austin, TX (US)

(21) Appl. No.: 15/485,005

(22) Filed: Apr. 11, 2017

Related U.S. Application Data
(60) Provisional application No. 62/321,479, filed on Apr. 12, 2016.

(51) Int. Cl. A61F 2/76; A61F 2/68; A61F 2/50

(52) U.S. Cl. CPC A61F 2/76; A61F 2/5046; A61F 2/68; A61F 2002/7695; A61F 2002/7665; A61F 2002/5056; A61F 2002/505

(57) ABSTRACT

A method utilizing digital scanning, additive manufacturing, and electronic embedded garments for advanced fitment of prosthetic devices.

### CROSS-REFERENCE TO RELATED APPLICATIONS

This application claims the priority of previously filed U.S. Provisional 62/321,479 filed Apr. 12, 2016.

### FIELD

This disclosure relates generally to advanced methodology for fitment of prosthetic devices.

### BACKGROUND

There exist over one million amputees living in North America who face significant challenges in acquiring a natural fit and range of motion when donning their prosthetic devices. Due to variations in individual anatomy and a lack of implemented technology, prosthetists can only engage in a labor-intensive and crude approach to fitment procedures without any measured feedback. Such procedures result in multiple return visits for adjustments, pain and discomfort for the patient, and in some cases, friction blisters or skin damage. The multi-million dollar cost associated with the negative effects of the current fitting process can be remedied with a specific arrangement of combined technologies for an innovative, cost-effective, and accurate solution to the fitment problem.

Prosthetists face significant challenges when designing the socket and suspension systems that hold prostheses on upper-limb amputees. Variations among individuals introduce unique complexities that factor into fitting the socket; these include muscle bundles, neuroma, bone spurs, and skin conditions such as scars from burns and sores from infections. Due to the difficulty of measuring socket interface characteristics without disturbing the secure fit of the socket, there is a lack of quantifiable diagnostic fitment information available to prosthetists. As a result, the process of fitting sockets is currently a labor-intensive, manual approach practiced by artisans. Current fitting techniques often yield sockets that are uncomfortable, unstable, or impede full range of motion, resulting in compromised device performance or election by the amputee to not use the prosthesis altogether.

The current fitment procedures implemented by prosthetists consist of two primary methods: plaster molds for non-weight bearing residual limbs and thermoplastic shaping for weight bearing limbs. The plaster method dictates a patient have plaster bandages placed upon their residual limb to collect an accurate representation of the limb shape. The bandages harden and an acceptable shape is collected. Similarly, for lower limb/weight bearing configurations, a thermoplastic shell approximately the shape of the distal end of the limb is applied to the patient. The shell is heated locally with an applied source (blow torch, heat gun, etc) in an attempt to map pressure to areas requiring greater control in the socket. However, what is not addressed in either of these fitment processes is tissue compliance.

Tissue compliance is the mechanical response of biologic material in the localized region under consideration. The mixture of muscle, bone, and fat leads to a unique set of properties that vary from point to point in their stiffness, restitution, and resilience. Taking compliance into consideration in the fitment procedures will allow the prosthetist have a complete picture of how best to shift high pressure points in the presence of bone to regions of higher muscle or fat content for enhanced comfort, fit, and control. A more uniquely distributed fit reduces discomfort in the prosthesis and extends the range of motion to the patient. Unfortunately, the current fitting procedure provides only crude approximations of tissue behavior with no numerical feedback on the exactness of fit.

Additionally, without compliance being considered during the fitting process, unique patient data is lost, which would ultimately provide enhanced fit, patient confidence in range of motion, a natural sensation associated with the socket interface, and greater overall limb health.

What is needed new fitting procedures (fitment) with compliance at the forefront of the design process.

### BRIEF SUMMARY OF THE CONCEPT

This need is addressed by the use of new fitting procedures with compliance at the forefront of the design process. Most component features of the traditional approach to fitment are abandoned. The proposed new procedure can be broken down into three primary components: residual limb scan, biometric correlation, and real time feedback.

The need is addressed by a method for new fitting procedures for prosthetic devices including at least: scanning of the prosthetic limb to obtain a point cloud representation of the prosthetic limb; creating a data file that defines a three-dimensional shape of the prosthetic limb; said data file further defining the boundaries of thin cross sectional regions of the prosthetic limb; providing the data file to an additive layer-wise manufacturing machine; producing a test socket of the prosthetic limb on the additive layer-wise manufacturing machine; obtaining biometric correlation using an electronics embedded garment applied to the prosthetic limb to capture data correlated to the geometry of the prosthetic limb; and analyzing the captured correlated data using three-dimensional reconstruction software to obtain real time pressure and temperature maps of the electronics embedded garment for use in the prosthetic fitting design; and applying the captured biometric data and the real time pressure and temperature maps from the electronics embedded garment to rapidly converge on a final fitment of the prosthetic device.

### DESCRIPTION OF THE FIGURES

FIG. 1 illustrates current artisanal method of casting a plaster mold of a residual limb

FIG. 2 illustrates the concept of scan, correlation, and feedback.

FIG. 3 illustrates the use of photogrammetry.

FIG. 4 illustrates the use of structured light scanning.

FIG. 5 is an example of the imaging possible from structured light scanning.

FIG. 6 illustrates an example meshing process for conversion of scanned data to a final representation.

FIG. 7 illustrates an E-Garment prosthetic sock.

FIG. 8 represents an example of a thin flexible printed circuit board that can be integrated into E-Garments.

FIG. 9 is an example of wearable flexible printed circuit board.

FIG. 10 is a black white representation of color mapping of a limb to represent regions of pressure.

FIG. 11 is an example of designed patterns (crossed over splines) in three layers of piezo-resistive fabric that can be used in E-garments.

FIG. 12 is a further example of designed patterns (crossed over splines) in three layers of piezo-resistive fabric that can be used in E-garments.

### DETAILED DESCRIPTION

In the following detailed description, reference is made to accompanying drawings that illustrate embodiments of the present disclosure. These embodiments are described in sufficient detail to enable a person of ordinary skill in the art to practice the disclosure without undue experimentation. It should be understood, however, that the embodiments and examples described herein are given by way of illustration only, and not by way of limitation. Various substitutions, modifications, additions, and rearrangements may be made without departing from the spirit of the present disclosure. Therefore, the description that follows is not to be taken in a limited sense, and the scope of the present disclosure will be defined only by the final claims.

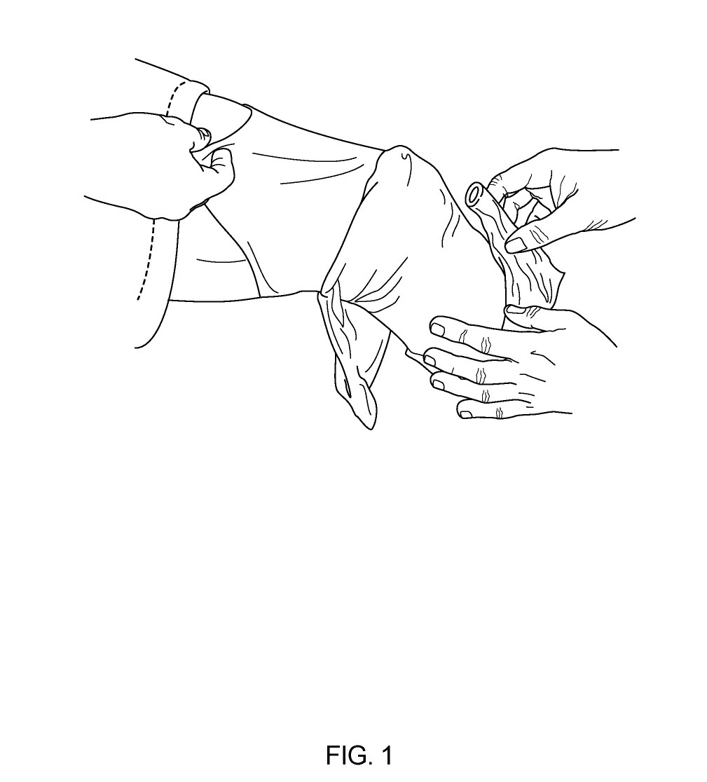

The current artisanal method of casting a plaster mold of the residual limb—is a cumbersome, messy, inaccurate, and fragile process. See FIG. 1. The laying on of plaster onto the patient requires two coveted resources: time and practice. The setting and curing process for the plaster can take anywhere between fifteen and sixty minutes, depending on the patient and the prosthetist. Understanding how to properly layer the plaster, with what consistency, material, and moisture content serves to underscore the artisanal requirements of this method.

The proposed replacement approach of this disclosure can be broken down into stages. Residual limb scan, creation of an accurate test socket using additive manufacturing, biometric correlation, and real time fitment feedback.

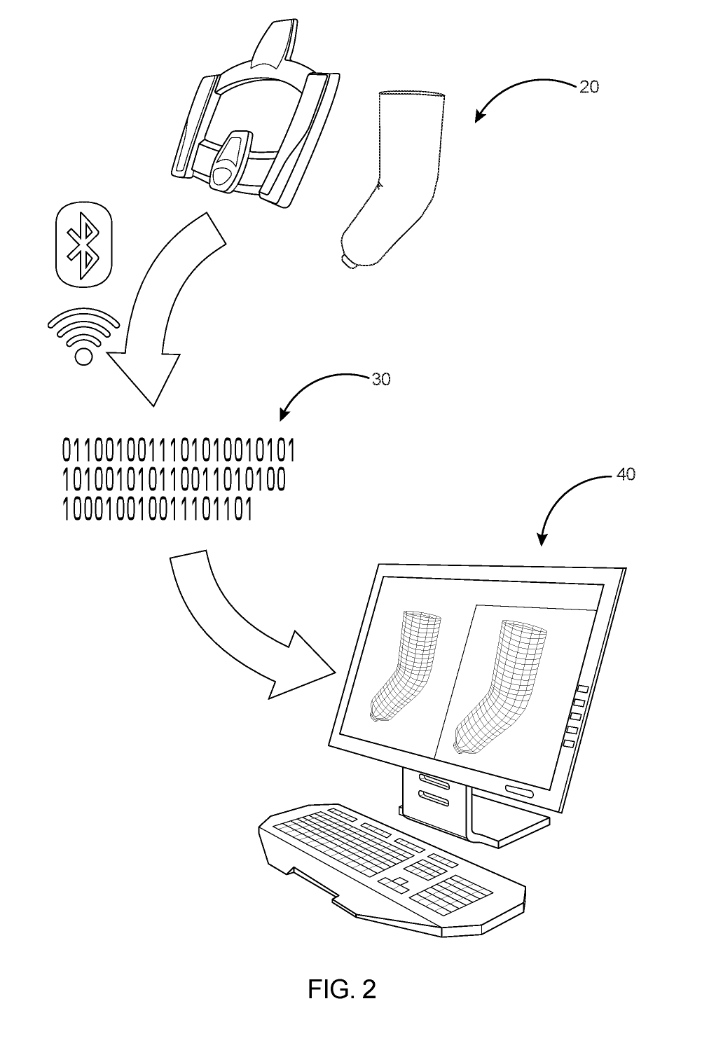

FIG. 2 illustrates the first stage of the method to be used. A prosthetic limb 20 is scanned using methodologies to be described and the digital data 30 is converted to digital images on a computer 40.

Whereas in conventional approaches the fit process is initiated with the use of an inconsistent plaster bandage application, the proposal herein begins with a three-dimensional residual limb scan. A number of technologies can be used for capturing three-dimensional images. These include Photogrammetry, LIDAR, conoscopic holography, and structured light programming for example. Each of these methods could be used in the process described herein. Two of these are described herein.

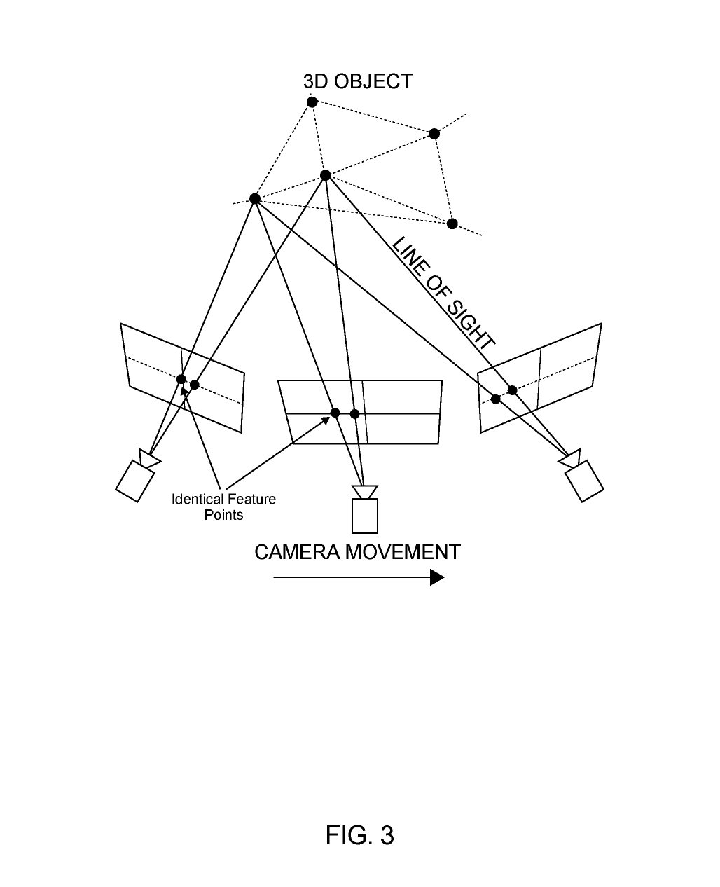

Using photogrammetry as the example embodiment, the process begins with standard photography of the residual limb from all angles (see FIG. 3). Photogrammetry is the science of making measurements from photographs, especially for recovering the exact positions of surface points. In Close-range Photogrammetry (CRP) the camera is close to the subject and is typically hand-held or on a tripod. Usually this type of photogrammetry is non-topographic—that is, the output is not topographic products Ike terrain models or topographic maps, but instead drawings, 3D models, measurements and point clouds. Everyday cameras can be used to model and measure a residual limb. This type of photogrammetry (CRP for short) is also sometimes called Image-Based Modeling. The cameras used are good quality digital cameras. As shown in FIG. 3 the camera and object to be scanned are moved relative to each other to systematically capture a surface cloud of points over the scanned object.

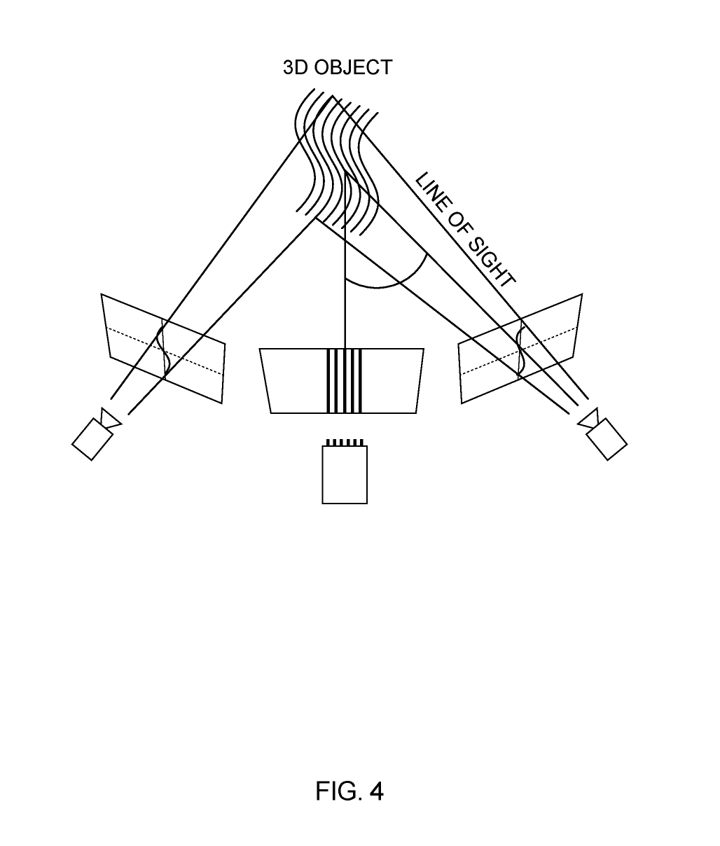

In an alternate embodiment Structured Light Scanning can be used (see FIG. 4). Structured Light Scanning is a fast, accurate, and versatile method for capturing an entire region of interest in the time it takes a camera flash to flash. An advantage over the photogrammetry described above is that photogrammetry requires a subject to remain still for the duration of the scan process, lest aberration or inaccuracies occur within the scan.

A structured-light 3D scanner is a 3D scanning device for measuring the three-dimensional shape of an object using projected light patterns and a camera system.

Projecting a narrow band of light onto a three-dimensional shaped surface using a stripe projector (as shown in FIG. 4) produces a line of illumination that appears distorted from other perspectives than that of the projector, and can be used for an exact geometric reconstruction of the surface shape. Horizontal and vertical light bands are projected on object surface and then captured by two webcams (cameras 1 and 2 in FIG. 4).

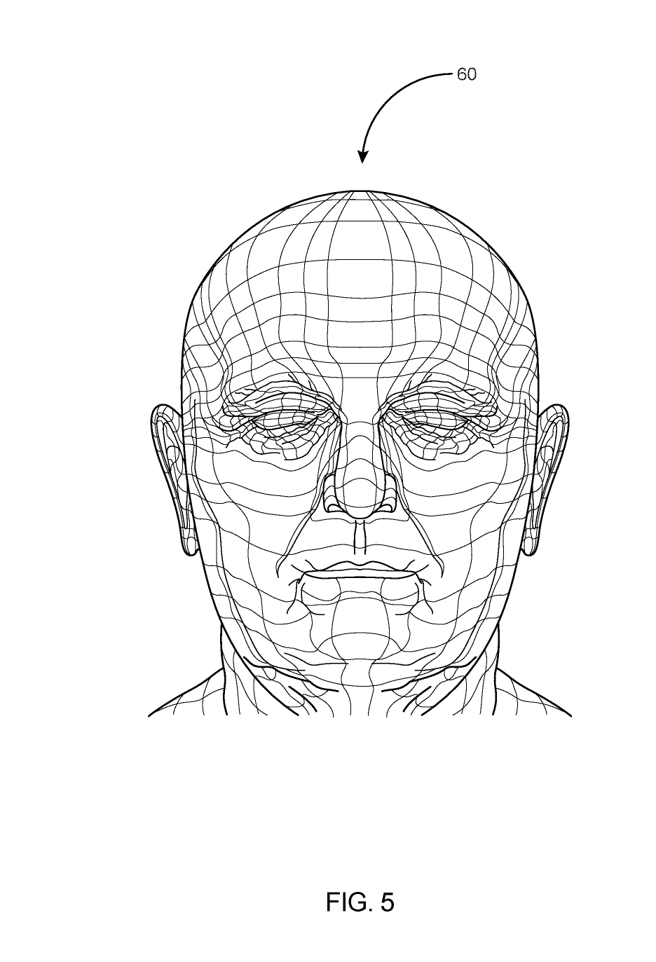

Structured light scanning can result in impressive detail in rendering human images, as seen in FIG. 5. However, structured light scanning can be more expensive than photogrammetry.

The use of either of these scanning techniques in the process is intended to outmode the current artisanal method of casting a plaster mold of the residual limb—a cumbersome, messy, inaccurate, and fragile process. The laying on of plaster onto the patient requires two coveted resources: time and practice. The setting and curing process for the plaster can take anywhere between fifteen and sixty minutes, depending on the patient and the prosthetist. Understanding how to properly layer the plaster, with what consistency, material, and moisture content serves to underscore the artisanal requirements of this method. The three-dimensional residual limb scan approach previously described is a significant improvement to the outdated approach of the use of plaster through the use of digital technology and the harnessing of computational power. The photographic reconstruction process only requires rudimentary operational knowledge of a camera and computer. The photogrammetry software process reconstructs the same digital version of the plaster mold in less than three minutes.

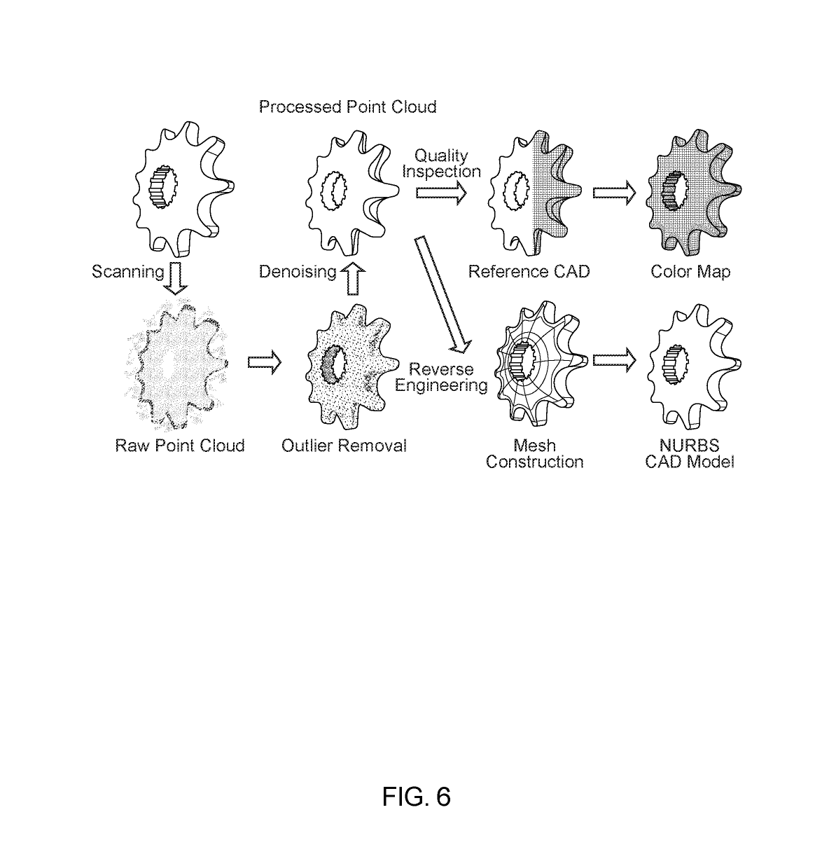

The raw point clouds then have trigonometric algorithms applied to them to create an amalgamation of the residual limb in digital space. Once this process is complete, a cloud point field is generated with specified distances and relationships between nearest neighbor points. This cloud field on its own is difficult to interpret but the data can be digitally modeled into either a polygonal model; which is a faceted (or tessellated) model consisting of many triangles (a “Standard Tessellation Language” or STL File) or some form of a Rapid NURBS model. In one embodiment the point cloud can be meshed with a Poisson Surface Reconstruction algorithm through CGAL, an open source C-based meshing utility. An example meshing process is shown in FIG. 6, which illustrates a typical flow process used in various scanning technologies to step through a process for converting the raw point cloud data from a scanning device eventually to a final NURBS CAD model. A number of commercially available software packages are available and it is anticipated any of them could be used to do this.

This meshed surface is part of what the prosthetist sees in a view on a monitor through the software. Current intuitive mouse controls allow for complete view manipulation with zoom, rotation, translation, and screen capture.

In an alternate embodiment the use of a monitor can be replaced by use of an augmented reality platform such as the GearVR recently released at CES 2015. It is a device wherein a Samsung smart phone (Galaxy s6 Edge and greater) can be inserted into a virtual reality headset for hands free display. This is especially critical to the prosthetist that must understand and intuit the state of the prosthetic interface with live representation and data.

At this point, under the artisanal approach, the plaster mold has dried and a series of steps occur in order to continue to the next significant stage: fitting of the test socket. These steps are to first create a negative mold, then a foam or silicone positive model, and a vacuum forming of the positive mold with thermoplastic. Two resources are required in great supply at this juncture: time and money. Typically, the patient is sent home after the plaster fitting for any number of days between two weeks and four months. A prosthetist with a multitude of patients at this point can become severely back-logged in generating test sockets and so needs to have a technical staff on hand or else send the plaster molds out to be manufactured into test sockets. If the process is decided to be done in house, the staff works with vacuum presses, silicon dispensing machinery, mills, and lathes. These pieces of equipment and staff are not without cost and is one that the prosthetist must absorb over years of time to sustain their business.

To circumvent the high cost of personnel, materials, and machinery an alternate pathway of approach is now used: printed test sockets prepared by the technology of layer-wise additive manufacturing, popularly called 3D Printing. A thermoplastic test socket can be prepared using additive manufacturing using a shelled surface model of the digitized limb geometry prepared from the scanning of the residual limb. The prosthetist would still have the ability to then shape the test socket before it is prepared for the building of the final socket. There are a variety of additive manufacturing technologies, including stereolithography, fused deposition modeling, and selective laser sintering, as well as others. The use of these technologies are available from a worldwide industry of additive manufacturing “service bureaus” that “build” 3-dimensional models from a variety of plastic or metal materials based on the aforementioned surface model of the digitized limb geometry prepared from scanning technologies such as photogrammetry or structured light scanning. Most of these additive manufacturing devices take in the previously described STL files for driving the layerwise additive manufacturing process.

The software within the machines then generates the layering instructions and directs the deposition of successive layers of material needed to build up the physical part. Essentially this part is created from cross sectional layers. The layers are fused together automatically and ultimately create the final shape, an exact physical replica of the 3D model. Additive manufacturing is an umbrella term that covers many of the following processes.
One of the earliest and most common types of AM is called Stereolithography (also known as SLA). SLA builds pieces using a laser and a vat of UV-curable liquid resin. Each thin layer of resin is solidified and secured to the layer below with every pass of the UV laser. SLA is good for producing models, patterns, and prototypes. A downside to SLA is that it generally requires support structures to be included in the build, which is part of the SLA process.
Another AM process is Selective Laser Sintering (also known as SLS). Unlike SLA, SLS can utilize a wide variety of materials such as plastics, metals, and ceramics although post processing may be required. SLS does not require support material while building since it is built and incased within the raw material. SLS uses these materials in a powder format and, by fusing the powder together, creates the layers needed to build the part. SLS is increasingly being used to create final parts for when mass scale production isn't necessary.
Similar to Stereolithography is Fused Deposition Modeling (also known as FDM). FDM also uses the additive platform build concept. Rather than raw liquid or powder, FDM uses thermoplastic materials that are applied through a heated nozzle that places a single thermoplastic bead at a time. These beads fuse together and harden as cooled. The plastics used in FDM are known for their strength and high heat resistance, making them good for product testing.

The result from the additive manufacturing step is a finished accurate three-dimensional model of the residual limb to be used as a test socket.

The availability of an accurate test socket obtained from a one time simple scan of the patients limb and the subsequent manufacture of the test socket from that data using additive manufacturing (3D Printing) provides an accurate socket to the designers during all of the remaining development period for fitting of various garments and other test equipment without having to repeatedly involve the patient in tedious fitting protocols of the previous artisanal methods.

In the proposed process described herein the next step is the creation of a biometric correlation between the residual limb scan and real time feedback.

The biometric correlation step in the overall process is done by making use of an E-garment or sensorized prosthetic garment platform. The garment is placed on the patient's residual limb after the test socket has been printed.

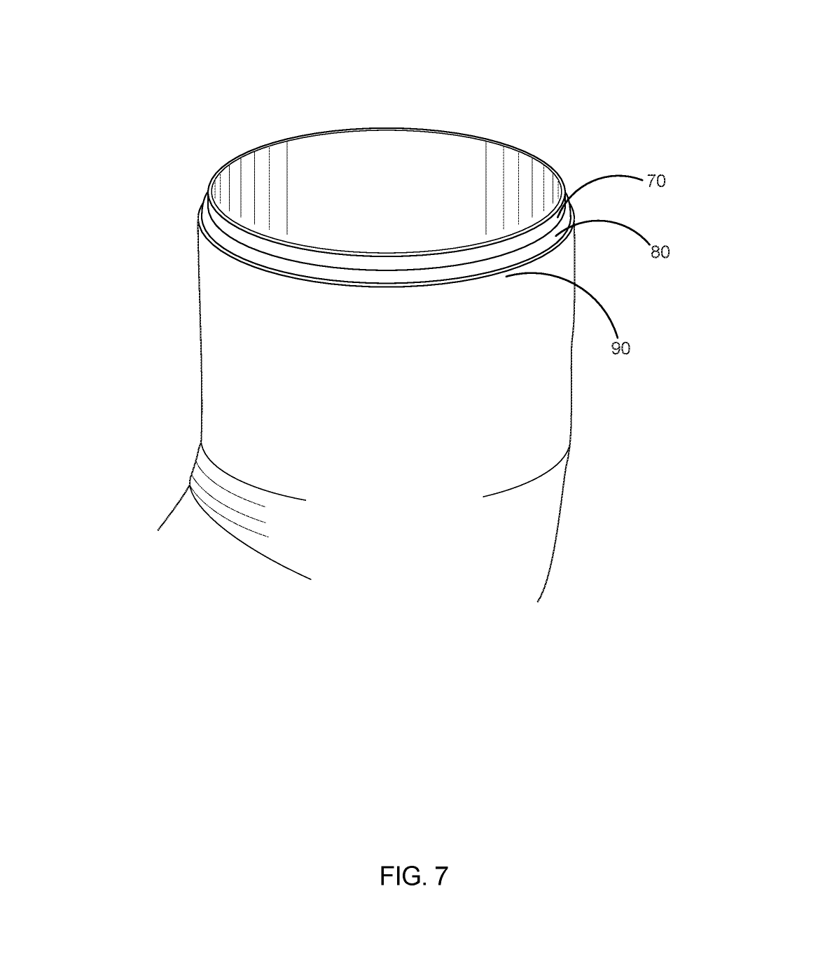

In one embodiment the E-garment can simply be a flexible electronics embedded garment similar to a prosthetic sock. FIG. 7 illustrates such an E-garment. An internal layer 70 may be of Goretex and cotton and an outer layer 90, may also be of Goretex and cotton. In-between those layers 80 are a sensor layer that can contain sensors encapsulated in thin layer printed circuit boards. These sensors capture data that can be correlated to the geometry of the residual limb by means of a coupled coordinate system.

The sensors required to do this could include:
A flexible piezo film element matrix (EMFIT ferro-electric thin film sensors)
Thin film RTD (Resistance Temperature Detector) elements (Innovative Sensor Technology)
Micro module Bluetooth controller (Murata Micro Bluetooth LBCA series)
A flexible PCB with micro elements to supply voltage regulation and other base functionality (EPEC flexible PCB manufacturers).

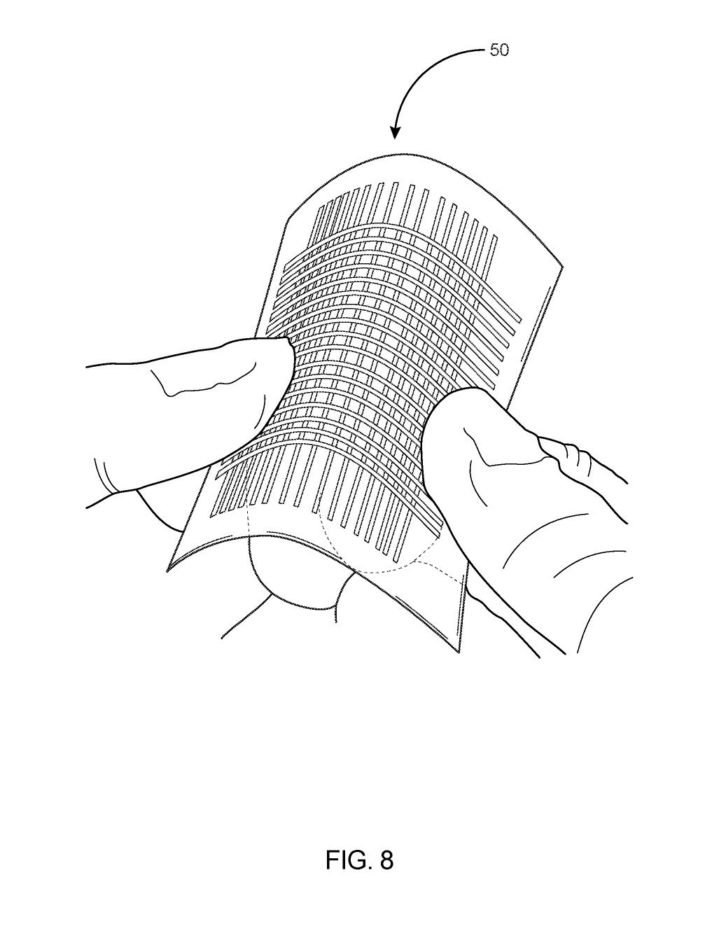

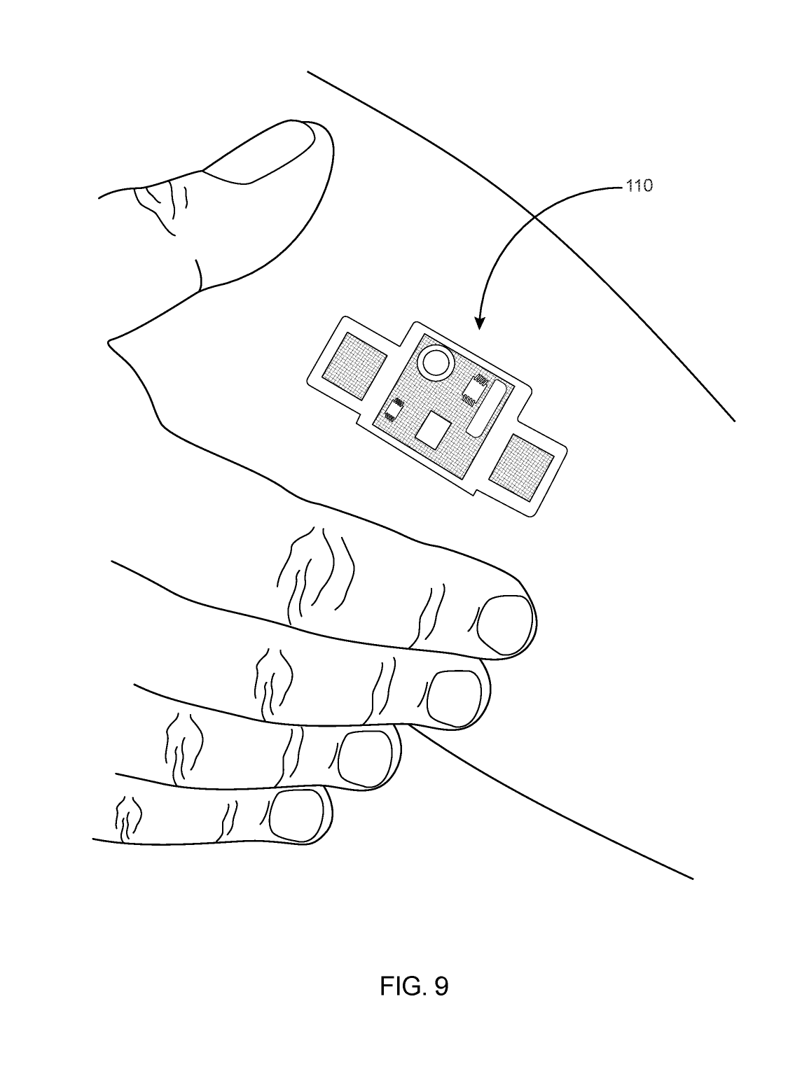

The EMFIT ferro-electric thin film sensors can be cut into any shape and still comprise a pressure sensitive sensor. Accordingly the manufacturing process is straightforward. FIGS. 8 and 9 are examples of such thin film sensors that can be used in a variety of ways in E-garments.

The flexible piezo film element matrices, thin film RTD's, Micro module Bluetooth controllers, or flexible PCB's will support the real time acquisition of pressure and temperature data across the interface of the fitting sock. Because the electronic elements are to be indirectly applied to the human body, it is required that they be of a flexible nature. Flexible PCBs are constructed from thin films of plastic substrate (polyimide or materials from the PEEK family) with surface mount components then mounted in a traditional manner (photolithography). The following components are required for the flexible PCB:
Bluetooth radio
Microcontroller unit
Multiplexed analog to digital converters
Voltage regulator
Multiplexed operational amplifiers
Various LRC components for stabilization of signals

Flexible tracks can be made from the multiplexed ADCs to the ferro-electric sensor array. Each track can handle multiple channels of data which will be optimized upon further discussion with a prosthetist on which zones bear greatest resolution of study. With a similar configuration applied to the posterior section of the E-garment for sensor array, the only difference is in lieu of a flexible PCB will be a flexible lithium based battery pack. Solicore Flexion series lithium batteries can be layered onto one another and connected in series to generate a higher capacity low voltage flexible power supply. These elements can similarly feature flexible tracks to meet the flexible PCB on the anterior side for a fully powered, wireless, 32 channel sensing element for prosthetic fitting.

The captured data from the E-garment can then be analyzed by 3 dimensional reconstruction software to examine pressure, temperature, and compliance. Example software that can supply this functionality is ReconstrucMe SDK developed in Austria by PROFACTOR. The output can then present color maps of pressure or temperature.

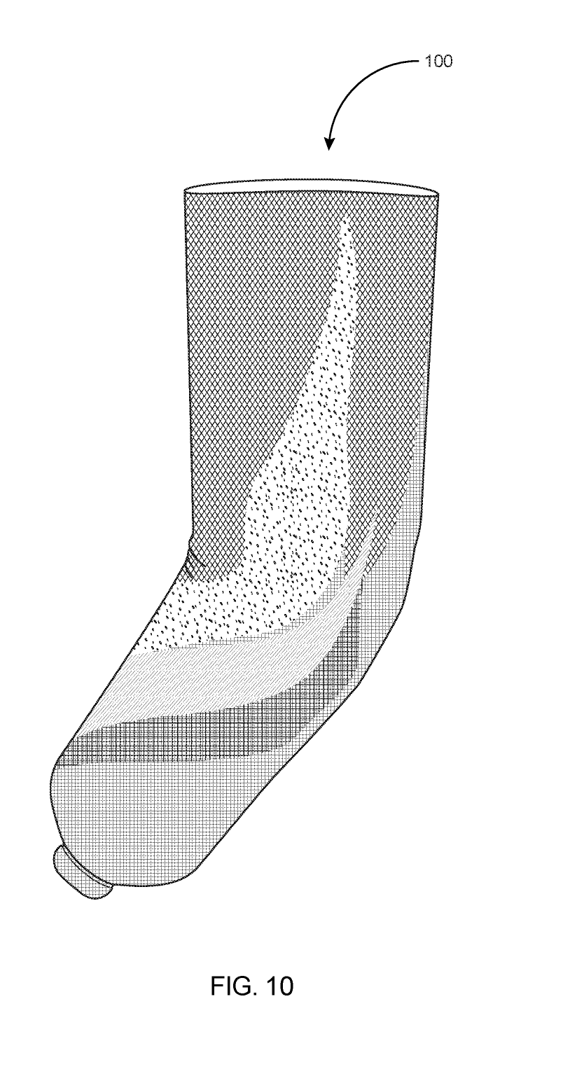

FIG. 10, represented generally by the numeral 100, is a black white representation of color mapping of a limb to represent regions of pressure using such 3-dimensional software. Similar plots can be generated for temperature.

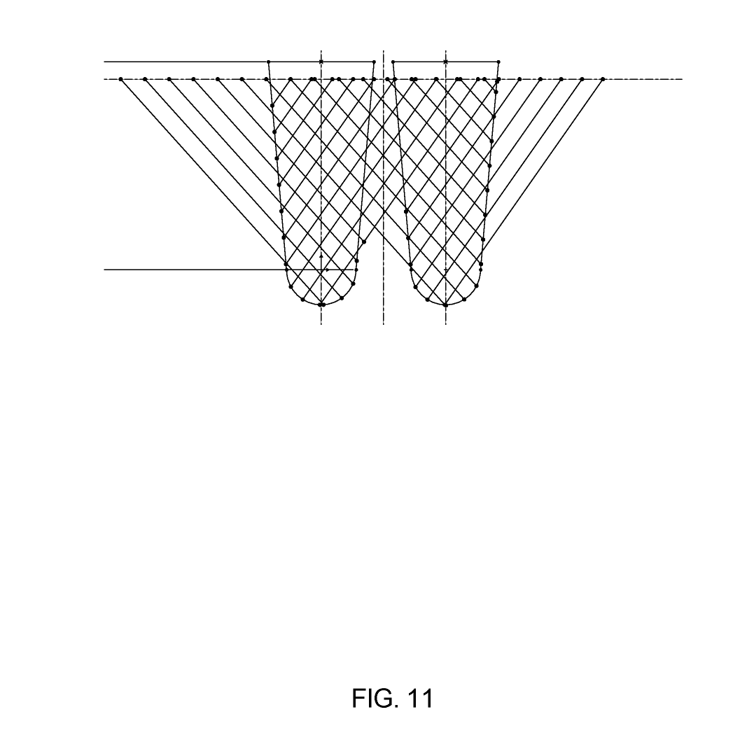

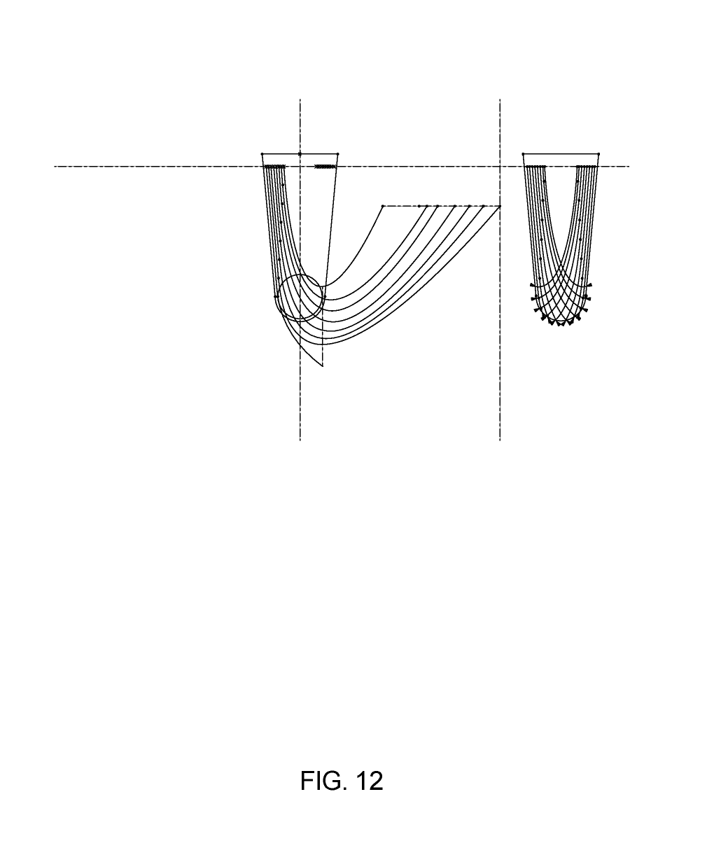

In an alternate embodiment the E-garment can be a sensorized prosthetic garment platform of carefully designed patterns (crossed over splines) in three layers of piezo-resistive fabric that responds electrically with respect to applied pressure. As this garment is powered and connected via USB to the computer hosting the software, data is gathered that can be mapped to the digital limb. These data are a direct representation of pressure on the limb surface. Examples of this approach can be seen in FIGS. 11 and 12. This approach addresses the most common and prevalent issue to both Myoelectric and analog prosthetic fitting: improper fit due to poor pressure distribution.

It was shown that the sensors respond approximately linearly with respect to an applied force. The mechanism of action that drives the change in resistance, and later voltage of a powered sensor, is the piezo-resistive fabric. The resolution of such a sensor is directly dependent on how tightly curves can be sewn close to one another. For example, in the three layer configuration, two of the layers contain lines sewn in of conductive thread (either stainless steel or silver-plated nylon). Each of these layers represents either a layer of “columns” or “rows”. Placed in between these layers is the piezo-resistive fabric. The lines for the row and column layers are drawn out and connected to a measuring source (oscilloscope, multi-meter, micro-controller, operational amplifier, etc). Then, all three layers are sewn together and a three-layer “patch” is the result. This patch can be of any shape desired and is only limited by the precision afforded by the machinery to construct it.

In either embodiment of E-garments the external data captured by the E-garment will be correlated to the geometry of the residual limb by means of a coupled coordinate system. When the scan of the limb is made, a physical target is placed on the limb to mark a coordinate origin.

Similarly, when the E-garment is applied, it will be oriented with its own coordinate origin at the same location as the scanning origin. This will ensure that both the scan and the garment data match on the digital representation of the limb. By removing the most cost-incurring and time consuming aspects of the fitment process with an immediate limb scan available for manipulation, the prosthetist can focus on socket optimization with the patient through real time feedback of the socket interface. The final aspect of the process to consider is fitment feedback. At this stage, compliance of the tissue will be considered.

It is at this stage where another bottleneck in the prosthetic design process occurs. In the conventional method for the fit process, as mentioned previously, a plaster cast is used and that cast is then sent off to a manufacturer to complete the socket design and is then sent back in the hope that the fit will be appropriate.

Typically, en route, the plaster mold can be lost, damaged, or otherwise have some critical information problem that makes it unsuitable, which results in a new cast being required.

With the application of the E-garment and the visual representation of the socket interface available to the prosthetist, the final fitting using the thermoplastic shell is dramatically enhanced. The prosthetist can now see, in real time, the levels of pressure on the interface to shape a drastically improved fit that will reduce re-visits, shipping hazards, and increase patient satisfaction. As a final measure of cost savings, the digital version of the socket interface becomes the new “mold of the limb.” This then becomes the product that the prosthetist and their manufacturers implement as their baseline of socket production.

The final socket production can be done in a variety of ways, but the availability of the detailed digital data from the method described herein offers also the production of the final socket from the application of additive manufacturing methods (3D Printing).

The net result of the three step process described herein will be a dramatically improved method of fitment. One that is not only more cost effective but a significant improvement in the overall experience for the amputee.

Although certain embodiments and their advantages have been described herein in detail, it should be understood that various changes, substitutions and alterations could be made without departing from the coverage as defined by the appended claims. Moreover, the potential applications of the disclosed techniques is not intended to be limited to the particular embodiments of the processes, machines, manufactures, means, methods and steps described herein. As a person of ordinary skill in the art will readily appreciate from this disclosure, other processes, machines, manufactures, means, methods, or steps, presently existing or later to be developed that perform substantially the same function or achieve substantially the same result as the corresponding embodiments described herein may be utilized. Accordingly, the appended claims are intended to include within their scope such processes, machines, manufactures, means, methods or steps.

1. A method for new fitting procedures for prosthetic devices comprising:
a. scanning of the prosthetic limb to obtain a point cloud representation of the prosthetic limb;
b. creating a data file that defines a three-dimensional shape of the prosthetic limb; said data file further defining the boundaries of thin cross sectional regions of the prosthetic limb;
c. providing the data file to an additive layer-wise manufacturing machine;
d. producing a test socket of the prosthetic limb on the additive layer-wise manufacturing machine;
e. obtaining biometric correlation using an electronics embedded garment applied to the prosthetic limb to capture data correlated to the geometry of the prosthetic limb;
f. analyzing the captured correlated data using three-dimensional reconstruction software to obtain real time pressure and temperature maps of the electronics embedded garment for use in the prosthetic fitting design; and
g. applying the captured biometric data and the real time pressure and temperature maps from the electronics embedded garment to rapidly converge on a final fitment of the prosthetic device.

2. The method for new fitting procedures for prosthetic devices of claim 1 wherein the scanning of the prosthetic limb uses photogrammetry for obtaining a point cloud representation of the prosthetic limb.

3. The method for new fitting procedures for prosthetic devices of claim 2 wherein a camera and the prosthetic limb are moved relative to each other to systematically capture a surface cloud of points over the prosthetic limb.

4. The method for new fitting procedures for prosthetic devices of claim 1 wherein the scanning of the prosthetic limb uses structured light scanning for obtaining a point cloud representation of the prosthetic limb.

5. The method for new fitting procedures for prosthetic devices of claim 4 wherein the structured light scanning projects horizontal and vertical bands of light onto the prosthetic limb and captures the resulting images using two cameras.

6. The method for new fitting procedures for prosthetic devices of claim 1 wherein the creating of a data file creates a Standard Tessellation Language or STL file.

7. The method for new fitting procedures for prosthetic devices of claim 1 wherein the providing of a data file to a layer-wise additive manufacturing machine is provided to a selective laser sintering (SLS) machine.

8. The method for new fitting procedures for prosthetic devices of claim 1 wherein the providing of a data file to a layer-wise additive manufacturing machine is provided to a stereolithography (SLA) machine.

9. The method for new fitting procedures for prosthetic devices of claim 1 wherein the providing of a data file to a layer-wise additive manufacturing machine is provided to a fused deposition modeling (FDM) machine.

10. The method for new fitting procedures for prosthetic devices of claim 1 wherein the electronics embedded garment applied to the prosthetic limb to capture data correlated to the geometry of the prosthetic limb contains an embedded flexible piezo film element matrix.

11. The method for new fitting procedures for prosthetic devices of claim 1 wherein the electronics embedded garment applied to the prosthetic limb to capture data correlated to the geometry of the prosthetic limb contains Thin film RTD (Resistance Temperature Detector) elements.

12. The method for new fitting procedures for prosthetic devices of claim 1 wherein the electronics embedded garment applied to the prosthetic limb to capture data correlated to the geometry of the prosthetic limb contains Micro module Bluetooth controllers.

13. The method for new fitting procedures for prosthetic devices of claim 1 wherein the electronics embedded garment applied to the prosthetic limb to capture data correlated to the geometry of the prosthetic limb contains flexible PCB with micro elements to supply voltage regulation and other base functionality.

14. The method for new fitting procedures for prosthetic devices of claim 1, wherein a final production socket is produced using the application of additive manufacturing methods.
